(19) United States
(12) Patent Application Publication
(10) Pub. No.: US 2026/0294281 A1
(43) Pub. Date: Oct. 1, 2026

(54) DETECTION DEVICE

(71) Applicant: Japan Display Inc., Tokyo (JP)

(72) Inventor: Kazuhide MOCHIZUKI, Tokyo (JP)

(21) Appl. No.: 19/678,585

(22) Filed: May 15, 2026

Related U.S. Application Data
(63) Continuation of application No. PCT/JP2024/040122, filed on Nov. 12, 2024.

(30) Foreign Application Priority Data
Nov. 22, 2023 (JP) 2023-198563

(51) Int. Cl. A61B 5/1171; A61B 5/00

(52) U.S. Cl. CPC A61B 5/1171; A61B 5/0059; A61B 5/6802

(57) ABSTRACT

According to an aspect, a detection device includes: a housing having a ring shape; a sensor substrate provided in the housing and curved along the shape of the housing; a first optical sensor and a second optical sensor provided to the sensor substrate; a light source disposed in the housing; and wiring lines coupling each of the first and second optical sensors to a terminal part provided to the sensor substrate. The sensor substrate has a first side extending in a first direction, a second side parallel to the first side, and a notch formed by cutting out the second side when the sensor substrate is developed into a flat shape. The notch includes a first portion having an arc shape with a curvature and is formed between the first and second optical sensors in the first direction. The light source is disposed in a region overlapping the notch.

### CROSS-REFERENCE TO RELATED APPLICATION

This application claims the benefit of priority from Japanese Patent Application No. 2023-198563 filed on Nov. 22, 2023 and International Patent Application No. PCT/JP2024/040122 filed on Nov. 12, 2024, the entire contents of which are incorporated herein by reference.

### BACKGROUND

### 1. Technical Field

What is disclosed herein relates to a detection device.

### 2. Description of the Related Art

Optical sensors capable of detecting fingerprint patterns and vein patterns are known (for example, Japanese Patent Application Laid-open Publication No. 2009-32005). Such optical sensors are incorporated into the housing of wearable devices, such as smartwatches, wristwatches, and wristbands, and are used to acquire biometric information, such as pulse waves. The optical sensors include a sensor substrate and a plurality of photodiodes mounted on the sensor substrate.

In a detection device with such an optical sensor, when the sensor substrate is deformed along the shape of the housing of the device, stress concentration may occur at a certain point in the sensor substrate. The stress concentration in the sensor substrate may cause wire breakage or degrade the detection performance of photodiodes.

For the foregoing reasons, there is a need for a detection device capable of reducing stress concentration in a sensor substrate.

### SUMMARY

According to an aspect, a detection device includes: a housing having a ring shape; a sensor substrate provided in the housing and curved along the shape of the housing; a first optical sensor and a second optical sensor provided to the sensor substrate; a light source disposed in the housing; and a plurality of wiring lines coupling each of the first optical sensor and the second optical sensor to a terminal part provided to the sensor substrate. The sensor substrate has a first side extending in a first direction, a second side parallel to the first side, and a notch formed by cutting out the second side when the sensor substrate is developed into a flat shape. The notch includes a first portion having an arc shape with a curvature throughout its whole length and is formed between the first optical sensor and the second optical sensor in the first direction. The light source is disposed in a region overlapping the notch.

According to an aspect, a detection device includes: a housing having a ring shape; a sensor substrate provided in the housing and curved along the shape of the housing; a first optical sensor and a second optical sensor provided to the sensor substrate; a light source disposed in the housing; and a plurality of wiring lines coupling each of the first optical sensor and the second optical sensor to a terminal part provided to the sensor substrate. The sensor substrate has a first side extending in a first direction, a second side parallel to the first side, and an opening formed between the first side and the second side when the sensor substrate is developed into a flat shape. A first width of the sensor substrate between the first side and an outer periphery of the opening facing the first side is larger than a second width of the sensor substrate between the second side and an outer periphery of the opening facing the second side. The opening is formed between the first optical sensor and the second optical sensor in the first direction. The light source is disposed in a region overlapping the opening.

### BRIEF DESCRIPTION OF THE DRAWINGS

FIG. 1 is a schematic view illustrating an exemplary external appearance when a state of a finger accommodated inside a detection device according to a first embodiment of the present disclosure is viewed from a lateral side of a housing;

FIG. 2 is a sectional view taken along II-II′ in FIG. 1;

FIG. 3 is a developed view illustrating a development example of a flexible substrate of the detection device according to the first embodiment;

FIG. 4 is a plan view illustrating a configuration example of a sensor substrate illustrated in FIG. 3;

FIG. 5 is a sectional view taken along V-V′ in FIG. 4;

FIG. 6 is a sectional view taken along VI-VI′ in FIG. 4;

FIG. 7 is a plan view illustrating a configuration example of the sensor substrate according to a first modification of the first embodiment;

FIG. 8 is a plan view illustrating a configuration example of the sensor substrate according to a second embodiment; and

FIG. 9 is a plan view illustrating a configuration example of the sensor substrate according to a second modification of the second embodiment.

### DETAILED DESCRIPTION

The following describes modes (embodiments) for carrying out the present disclosure in detail with reference to the drawings. The present disclosure is not limited to the description of the embodiments given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the present disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present disclosure and the drawings, and detailed description thereof may not be repeated where appropriate.

In the present disclosure, in expressing an aspect of disposing another structure on or above a certain structure, a case of simply expressing “on” includes both a case of disposing the other structure immediately on the certain structure so as to contact the certain structure and a case of disposing the other structure above the certain structure with still another structure interposed therebetween, unless otherwise specified.

### First Embodiment

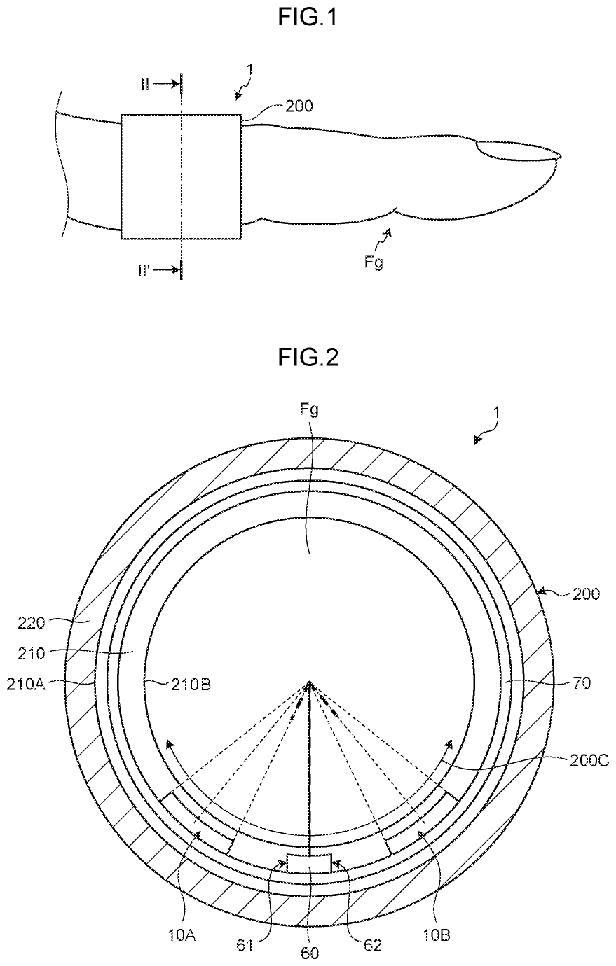

FIG. 1 is a schematic view illustrating an exemplary external appearance when a state of a finger accommodated inside a detection device according to a first embodiment of the present disclosure is viewed from a lateral side of a housing. FIG. 2 is a sectional view taken along II-II′ in FIG. 1.

As illustrated in FIG. 1, a detection device 1 according to the first embodiment is a finger ring-shaped (ring-type) device that can be worn on and removed from a human body. The detection device 1 is worn, for example, on a finger Fg of the human body. Examples of the finger Fg include a thumb, an index finger, a middle finger, a ring finger, and a little finger. The human body is a person to be authenticated whose identity is to be verified by the detection device 1. The detection device 1 can detect biometric information on a living body from the finger Fg wearing the detection device 1. The finger Fg is an example of a measurement target. The measurement target is the living body or a part of the living body, and is an object to be measured. The detection device 1 is formed as a finger ring or a wristband so as to be easily carried by a user. In the following description, the detection device 1 is assumed to be used as a finger ring.

While the detection device 1 is a ring-shaped device, it is not limited thereto. The detection device 1 may be incorporated into a wristwatch or a wristband and configured as a wearable device.

As illustrated in FIG. 2, the detection device 1 includes a housing 200, a light source 60, a first optical sensor 10A, a second optical sensor 10B, and a flexible printed circuit board 70. The detection device 1 includes a battery (not illustrated) inside the housing 200 and is operated by power of the battery.

The housing 200 is a wearable member that is formed in a ring shape (annular shape) wearable on the finger Fg, and is worn on the living body. As illustrated in FIG. 2, the housing 200 includes a first housing 210 and a second housing 220. The first housing 210 is integrated with the second housing 220 to form the housing 200 into the ring shape.

The first housing 210 is a member that contacts the human body on which the housing 200 is worn. The first housing 210 accommodates therein the light source 60, the first optical sensor 10A, the second optical sensor 10B, and other components. The first housing 210 is formed into a ring shape using a housing material, such as a light-transmitting synthetic resin or silicone.

The second housing 220 has a surface of the housing 200 that covers an outer peripheral surface 210A of the first housing 210. The second housing 220 is formed into a ring shape using a member of, for example, a metal or a non-light-transmitting synthetic resin. The first housing 210 of the housing 200 accommodates the flexible printed circuit board 70 on which the light source 60, the first optical sensor 10A, the second optical sensor 10B, and other components are formed. The flexible printed circuit board 70 is accommodated in the housing 200, for example, by being formed into a ring shape and, in that state, filling a periphery thereof with a filling material in a mold to form the housing 200.

The first optical sensor 10A and the second optical sensor 10B according to the present embodiment are provided so as to sandwich the light source 60 in a circumferential direction 200C. In other words, the first optical sensor 10A, the light source 60, and the second optical sensor 10B of the detection device 1 are arranged in this order in the circumferential direction 200C. By being arranged so as to sandwich the light source 60 in the circumferential direction 200C, the first optical sensor 10A and the second optical sensor 10B can detect light emitted from the light source 60 over a wide area of the housing 200.

The first optical sensor 10A and the second optical sensor 10B detect light emitted by the light source 60 and reflected by the finger Fg or the like, light directly incident on the optical sensor, and other light. The first optical sensor 10A and the second optical sensor 10B are organic photodiodes (OPDs). The first optical sensor 10A is provided to the housing 200 so as to be adjacent to a first end 61 of the light source 60 in the circumferential direction 200C of the housing 200. The second optical sensor 10B is provided to the housing 200 so as to be adjacent to a second end 62 of the light source 60 in the circumferential direction 200C of the housing 200.

As illustrated in FIG. 2, the light source 60 is provided in the first housing 210 of the housing 200, and is capable of emitting light toward the finger Fg wearing the housing 200. For example, inorganic light-emitting diodes (LEDs) or organic electroluminescent (EL) diodes (organic light-emitting diodes (OLEDs)) are used as the light source 60. The light source 60 emits light having a predetermined wavelength. In the present embodiment, the light source 60 includes a plurality of light sources so as to be capable of emitting near-infrared light, red light, and green light.

Light emitted from the light source 60 is reflected on the surface of an object to be detected, such as the finger Fg, and enters the first optical sensor 10A and the second optical sensor 10B. As a result, the detection device 1 can detect a fingerprint by detecting a shape of asperities on the surface of the finger Fg or the like. Alternatively, the light emitted from the light source 60 may be reflected in the finger Fg or the like or transmitted through the finger Fg or the like and enter the first optical sensor 10A and the second optical sensor 10B. Thus, the detection device 1 can detect information on a living body in the finger Fg or the like. Examples of the information on the living body include, but are not limited to, pulse waves, pulsation, and a vascular image of the finger or a palm. That is, the detection device 1 may be configured as a fingerprint detection device to detect the fingerprint or a vein detection device to detect a vascular pattern of, for example, veins.

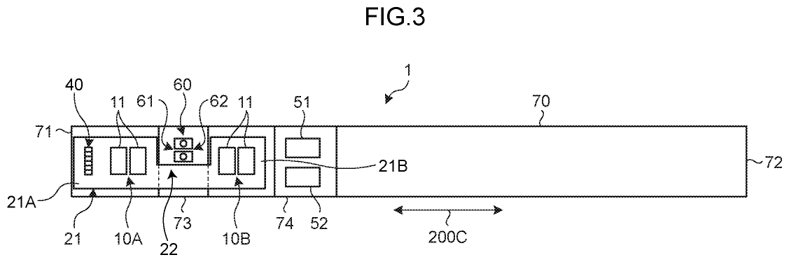

FIG. 3 is a developed view illustrating a development example of the flexible substrate of the detection device according to the first embodiment. As illustrated in FIG. 3, the flexible printed circuit board 70 has a deformable strip shape and is formed into a ring shape by coupling a first end 71 to a second end 72. The flexible printed circuit board 70 has a first fabrication region 73 and a second fabrication region 74. The first fabrication region 73 is a region on which the light source 60 and other components are fabricated. The second fabrication region 74 is a region on which a control circuit 51, a power supply circuit 52, and other components are fabricated.

A sensor substrate 21 is mounted on the flexible printed circuit board 70 so as to partially surround the light source 60 in the first fabrication region 73. The sensor substrate 21 is provided with the first optical sensor 10A, the second optical sensor 10B, and other components. The sensor substrate 21 is a deformable insulating substrate formed into a strip shape made of, for example, a film-like resin. The sensor substrate 21 is provided inside the housing 200 and is curved along the shape of the housing 200. The flexible printed circuit board 70 electrically couples the first optical sensor 10A and the second optical sensor 10B on the sensor substrate 21, the light source 60, and the control circuit 51.

In the present embodiment, as illustrated in FIG. 2, the flexible printed circuit board 70 is accommodated in the housing 200 such that the surface provided with the first optical sensor 10A, the second optical sensor 10B, and the light source 60 is positioned on the inner peripheral side of the housing 200. If the flexible printed circuit board 70 has a light-transmitting property, the first optical sensor 10A, the second optical sensor 10B, and the light source 60 may be mounted on the back surface opposite the surface. In this case, the light source 60 only needs to be arranged so that light is emitted toward the flexible printed circuit board 70 and light transmitted through the flexible printed circuit board 70 is emitted toward the outside of the housing 200.

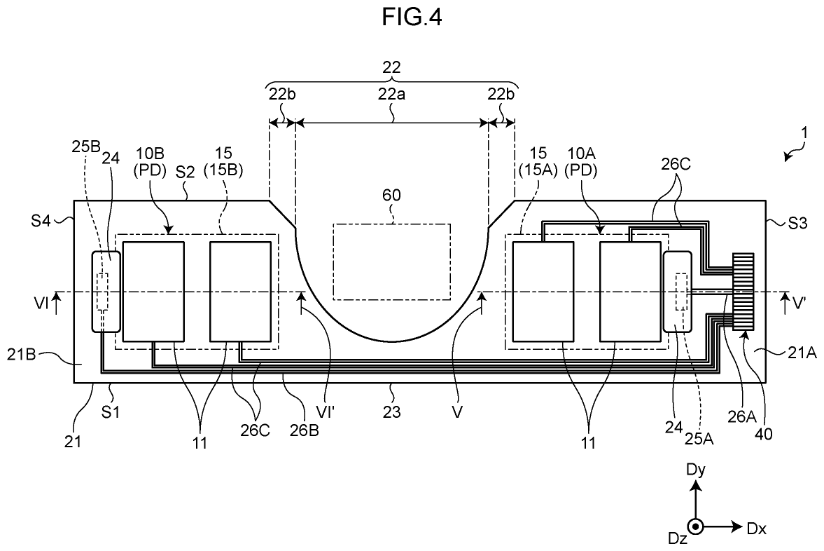

The following describes the configuration of the sensor substrate in detail with reference to FIG. 4. FIG. 4 is a plan view illustrating a configuration example of the sensor substrate illustrated in FIG. 3. FIG. 4 is a plan view of the sensor substrate 21 when it is developed into a flat shape.

In the following description, a first direction Dx is one direction in a plane parallel to the sensor substrate 21. A second direction Dy is one direction in the plane parallel to the sensor substrate 21 and is a direction orthogonal to the first direction Dx. The second direction Dy may non-orthogonally intersect the first direction Dx. A third direction Dz is a direction orthogonal to the first direction Dx and the second direction Dy. The third direction Dz is a direction normal to the sensor substrate 21. The term “plan view” refers to a positional relation as viewed from a direction perpendicular to the sensor substrate 21.

As illustrated in FIG. 4, the sensor substrate 21 has a notch 22 between two ends in the circumferential direction 200C of the housing 200, that is, in the longitudinal direction (first direction Dx) of the sensor substrate 21. The sensor substrate 21 is provided with the first optical sensor 10A on a first end 21A side and the second optical sensor 10B on a second end 21B side with the notch 22 interposed therebetween. In other words, the notch 22 is positioned between the first optical sensor 10A and the second optical sensor 10B in the first direction Dx. A terminal part 40 is provided at the first end 21A in the longitudinal direction of the sensor substrate 21. The terminal part 40 supplies power from the power supply circuit 52 (refer to FIG. 3) to the first optical sensor 10A and the second optical sensor 10B.

The sensor substrate 21 includes a connecting portion 23 corresponding to the portion where the notch 22 is formed. The connecting portion 23 is in contact with the notch 22 and connects the region of the sensor substrate 21 provided with the first optical sensor 10A and the region provided with the second optical sensor 10B. As a result, the region of the sensor substrate 21 provided with the first optical sensor 10A and the region provided with the second optical sensor 10B are integrally formed.

More specifically, the sensor substrate 21 has a first side S1 extending in the first direction Dx, a second side S2 extending in a direction parallel to the first side S1, and a third side S3 and a fourth side S4 extending in the second direction Dy between the first side S1 and the second side S2. The notch 22 is formed by cutting out the second side S2 of the sensor substrate 21.

The notch 22 has a first portion 22a and second portions 22b. The notch 22 is composed of the second portion 22b, the first portion 22a, and the second portion 22b formed in this order in the first direction Dx. The first portion 22a has an arc shape with a curvature throughout its whole length. More specifically, in the first portion 22a, the outer periphery of the sensor substrate 21 is formed into a recessed shape recessed from the second side S2 toward the first side S1, and has a continuous curved shape from one end to the other end in the first direction Dx. More preferably, in the first portion 22a, the outer periphery of the sensor substrate 21 has substantially no linear portion. The curvature of the first portion 22a of the notch 22 is, for example, approximately 1/(4×10⁻³) to 1/(3×10⁻³) rad/m.

Each second portion 22b is formed between the first portion 22a and the second side S2. More specifically, one of the second portions 22b is formed between one end of the first portion 22a and the second side S2 on the first end 21A side of the sensor substrate 21. The other of the second portions 22b is formed between the other end of the first portion 22a and the second side S2 on the second end 21B side of the sensor substrate 21. Each second portion 22b extends at an angle in a direction different from the tangential direction at the corresponding end of the first portion 22a.

The light source 60 is disposed in the region overlapping the notch 22. The notch 22 is formed to have a length in the first direction Dx longer than the length of the light source 60. The notch 22 is formed to have a length in the second direction Dy longer than the length of the light source 60 and shorter than the length (width) of the sensor substrate 21. More specifically, the distance in the first direction Dx between one end in the first direction Dx of the first portion 22a and the other end in the first direction Dx is longer than the length of the light source 60 in the first direction Dx. The distance in the second direction Dy between the center in the first direction Dx of the first portion 22a and the second side S2 of the sensor substrate 21 is longer than the length of the light source 60 in the second direction Dy. With this configuration, the notch 22 of the sensor substrate 21 can secure a space for disposing the light source 60.

In the sensor substrate 21 according to the present embodiment, the notch 22 is formed in a continuous arc shape. Therefore, the width in the second direction Dy of the connecting portion 23 corresponding to the notch 22 varies continuously. The connecting portion 23 has the smallest width at the center in the first direction Dx. The width of the connecting portion 23 in the second direction Dy increases continuously from the center in the first direction Dx toward the first optical sensor 10A or the second optical sensor 10B. This configuration can reduce local stress concentration in the sensor substrate 21 when the sensor substrate 21 is curved into a ring shape along the shape of the housing 200, compared with a configuration in which the notch 22 is not entirely in the form of a continuous arc and is formed, for example, in a rectangular shape.

In the example illustrated in FIG. 4, the stress is likely to be larger at the center in the first direction Dx of the first portion 22a, that is, the portion where the width in the second direction Dy of the connecting portion 23 is the smallest. Also in this case, since the notch 22 is formed in a continuous arc shape, the stress is distributed along the arc of the notch 22, and the stress generated at the center in the first direction Dx of the first portion 22a can be reduced.

Since the notch 22 has the second portion 22b, no corner is formed between the end of the notch 22 and the second side S2 of the sensor substrate 21. Therefore, when the sensor substrate 21 is deformed into a ring shape and accommodated in the housing 200, the portion of the sensor substrate 21 corresponding to the second portion 22b can be smoothly deformed along the shape of the housing 200, compared with a case where the second portion 22b is not formed and the end of the notch 22 and the second side S2 of the sensor substrate 21 are coupled substantially at a right angle. This configuration can reduce the stress at the end of the notch 22 on the second side S2 of the sensor substrate 21.

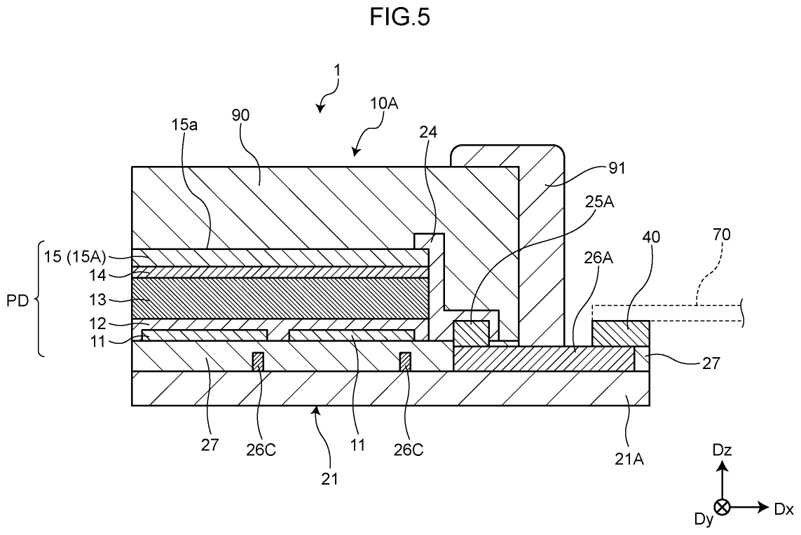

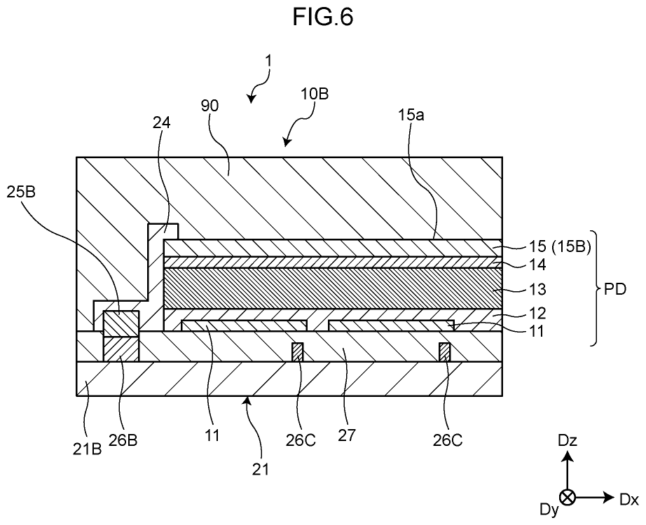

Next, the configurations of the first optical sensor 10A and the second optical sensor 10B are described with reference to FIGS. 4 to 6. FIG. 5 is a sectional view taken along V-V′ in FIG. 4. FIG. 6 is a sectional view taken along VI-VI′ in FIG. 4.

As illustrated in FIG. 4, the first optical sensor 10A has a multilayered configuration in which one upper electrode 15A covers two lower electrodes 11 arranged in the first direction Dx. The second optical sensor 10B has a multilayered configuration in which one upper electrode 15B covers two lower electrodes 11 arranged in the first direction Dx. An upper electrode 15 includes the upper electrode 15A of the first optical sensor 10A and the upper electrode 15B of the second optical sensor 10B. The upper electrode 15A and the upper electrode 15B have a rectangular surface and are independent electrodes that are not electrically coupled.

A first power supply electrode 25A and a second power supply electrode 25B are provided on the sensor substrate 21 and extend along the second direction Dy. The first power supply electrode 25A is provided between the first end 21A in the first direction Dx of the sensor substrate 21 and the first optical sensor 10A. The second power supply electrode 25B is provided between the second end 21B in the first direction Dx of the sensor substrate 21 and the second optical sensor 10B. The first power supply electrode 25A is electrically coupled to the terminal part 40 of the sensor substrate 21 via a first wiring line 26A and supplied with power signals from the power supply circuit 52 (refer to FIG. 3) via the terminal part 40. The second power supply electrode 25B is electrically coupled to the terminal part 40 of the sensor substrate 21 via a second wiring line 26B and supplied with power signals from the power supply circuit 52 via the terminal part 40.

The upper electrode 15A of the first optical sensor 10A is coupled to the first power supply electrode 25A via a conductive member 24 and electrically coupled to the terminal part 40 via the first wiring line 26A coupled to the first power supply electrode 25A. The upper electrode 15B of the second optical sensor 10B is coupled to the second power supply electrode 25B via a conductive member 24 and electrically coupled to the terminal part 40 via the second wiring line 26B coupled to the second power supply electrode 25B. As a result, the upper electrode 15A and the upper electrode 15B are supplied with power from the independent power systems of the first power supply electrode 25A and the second power supply electrode 25B, respectively. Each of the conductive members 24 is formed of a conductive material and covers the entire surface of the first power supply electrode 25A or the second power supply electrode 25B. The conductive member 24 electrically couples the first power supply electrode 25A to the upper electrode 15A or the second power supply electrode 25B to the upper electrode 15B. The upper electrode 15A and the upper electrode 15B may be directly coupled to the first power supply electrode 25A and the second power supply electrode 25B, respectively, without the conductive member 24.

Third wiring lines 26C each couple each of the lower electrodes 11 of the first optical sensor 10A or the second optical sensor 10B to the corresponding terminal part 40 provided to the sensor substrate 21. The third wiring line 26C coupled to the lower electrode 11 of the first optical sensor 10A is coupled to the terminal part 40 through a region on the second side S2 side of the sensor substrate 21. The third wiring line 26C coupled to the lower electrode 11 of the second optical sensor 10B is coupled to the terminal part 40 through a region on the first side S1 side of the sensor substrate 21 and the connecting portion 23. The third wiring lines 26C are coupled to a detection circuit of the control circuit 51 via the terminal part 40 and signal lines of the flexible printed circuit board 70. In other words, the detection circuit of the control circuit 51 is electrically coupled to the lower electrodes 11 of the first optical sensor 10A and the second optical sensor 10B via the signal lines. The detection circuit may be formed as a separate circuit from the control circuit 51.

The first power supply electrode 25A and the second power supply electrode 25B are supplied with power signals from the power supply circuit 52 via the terminal part 40 and supply power signals to the upper electrode 15A and the upper electrode 15B. In the example illustrated in FIG. 4, the first power supply electrode 25A and the second power supply electrode 25B are formed in a substantially rectangular shape extending in the second direction Dy in plan view and have the same area (size).

As illustrated in FIG. 5, a photodiode PD included in the first optical sensor 10A is provided on the sensor substrate 21 with an insulating layer 27 interposed therebetween.

The third wiring lines 26C are provided on the upper surface of the sensor substrate 21. The third wiring lines 26C are formed, for example, of metal lines, and are formed of a material having better conductivity than the lower electrodes 11 of the first optical sensor 10A. The third wiring lines 26C are provided in a layer between the sensor substrate 21 and the photodiode PD in the third direction Dz. The third wiring lines 26C are electrically coupled to the terminal part 40 on the sensor substrate 21 (refer to FIG. 4). The third wiring lines 26C may be formed, for example, in the same layer as the lower electrodes 11, and/or may be formed of metal. The insulating layer 27 is provided on the sensor substrate 21 to cover the third wiring lines 26C. The insulating layer 27 may be an inorganic insulating film or an organic insulating film.

The photodiode PD is provided as a sensor element on the insulating layer 27. The photodiode PD includes the lower electrode 11, a lower buffer layer 12, an active layer 13, an upper buffer layer 14, and the upper electrode 15 (upper electrode 15A). In the photodiode PD, the lower electrode 11, the lower buffer layer 12, the active layer 13, the upper buffer layer 14, and the upper electrode 15 are stacked in this order in the third direction Dz perpendicular to the sensor substrate 21.

The lower electrode 11 is formed, for example, of a light-transmitting conductive material such as indium tin oxide (ITO).

The active layer 13 changes in characteristics (for example, voltage-current characteristics and resistance value) depending on light emitted thereto. An organic material is used as a material of the active layer 13. Specifically, the active layer 13 has a bulk heterostructure containing a mixture of a p-type organic semiconductor and an n-type fullerene derivative ((6,6)-phenyl-C₆₁-butyric acid methyl ester (PCBM)) that is an n-type organic semiconductor. As the active layer 13, low-molecular-weight organic materials can be used including, for example, fullerene (C₆₀), phenyl-C₆₁-butyric acid methyl ester (PCBM), copper phthalocyanine (CuPc), fluorinated copper phthalocyanine (F₁₆CuPc), 5,6,11,12-tetraphenyltetracene (rubrene), and perylene diimide (PDI) (derivative of perylene).

The active layer 13 can be formed by a vapor deposition process (dry process) using any of the low-molecular-weight organic materials listed above. In this case, the active layer 13 may be, for example, a multilayered film of CuPc and F₁₆CuPc, or a multilayered film of rubrene and C₆₀. The active layer 13 can also be formed by a coating process (wet process). In this case, the active layer 13 is made using a material obtained by combining any of the above-listed low-molecular-weight organic materials with a high-molecular-weight organic material. As the high-molecular-weight organic material, for example, poly(3-hexylthiophene) (P3HT) and F8-alt-benzothiadiazole (F8BT) can be used. The active layer 13 can be a film made of a mixture of P3HT and PCBM, or a film made of a mixture of F8BT and PDI.

The lower buffer layer 12 and the upper buffer layer 14 are provided to facilitate holes and electrons generated in the active layer 13 to reach the lower electrode 11 or the upper electrode 15. One of the lower buffer layer 12 and the upper buffer layer 14 is a hole transport layer. The other of the lower buffer layer 12 and the upper buffer layer 14 is an electron transport layer. The material of the hole transport layer is a metal oxide layer. For example, tungsten oxide (WO₃) or molybdenum oxide is used as the metal oxide layer. Polyethylenimine ethoxylated (PEIE) is used as a material of the electron transport layer.

The materials and the manufacturing methods of the lower buffer layer 12, the active layer 13, and the upper buffer layer 14 are merely exemplary, and other materials and manufacturing methods may be used. For example, each of the lower buffer layer 12 and the upper buffer layer 14 is not limited to a single-layer film, and may be formed as a multilayered film that includes an electron blocking layer and a hole blocking layer.

The upper electrode 15 is provided on the upper buffer layer 14. The upper electrode 15 is continuously formed over the entire photodiode PD of the first optical sensor 10A. In other words, the upper electrode 15 is continuously provided on a plurality of photodiodes PD. The upper electrode 15 faces the lower electrodes 11 with the lower buffer layer 12, the active layer 13, and the upper buffer layer 14 interposed therebetween. The upper electrode 15 is formed, for example, of a light-transmitting conductive material such as ITO or indium zinc oxide (IZO). A portion of an end of an upper surface 15a of the upper electrode 15 is electrically coupled to the conductive member 24. The conductive member 24 is electrically coupled to the first power supply electrode 25A and supplies the power signals from the first power supply electrode 25A to the upper electrode 15.

A sealing film 90 is provided on the upper electrode 15. The sealing film 90 is an inorganic film, such as a silicon nitride film and an aluminum oxide film, or a resin film made of acrylic. The sealing film 90 is not limited to a single layer, and may be a multilayered film composed of two or more layers obtained by combining the inorganic film and the resin film described above. The sealing film 90 satisfactorily seals the photodiode PD and inhibits moisture from entering from the upper surface side. The photodiode PD according to the present embodiment is covered by a resin 91 from the sealing film 90 to part of the terminal part 40 to protect the terminal part 40, the sensor substrate 21, and other components.

As illustrated in FIG. 6, the two lower electrodes 11 of the second optical sensor 10B are provided in a different region of the sensor substrate 21 from the lower electrodes 11 of the first optical sensor 10A. The lower electrode 11 is covered by the lower buffer layer 12, the active layer 13, the upper buffer layer 14, and the upper electrode 15B. The photodiode PD included in the second optical sensor 10B has the same configuration as the photodiode PD of the first optical sensor 10A described above. In other words, the photodiode PD of the second optical sensor 10B includes the lower electrode 11, the lower buffer layer 12, the active layer 13, the upper buffer layer 14, and the upper electrode 15B. The first optical sensor 10A and the second optical sensor 10B according to the present embodiment are organic photodiodes.

In the second optical sensor 10B, a portion of an end of the upper surface 15a of the upper electrode 15 is electrically coupled to the conductive member 24, and the conductive member 24 is electrically coupled to the second power supply electrode 25B. The second optical sensor 10B supplies the power signals from the second power supply electrode 25B to the upper electrode 15. In the second optical sensor 10B, the sealing film 90 is provided on the upper electrode 15, the conductive member 24, and other components to satisfactorily seal the photodiode PD.

As described above, the sensor substrate 21 according to the present embodiment has the notch 22 that is entirely in the form of a continuous arc. With this configuration, the present embodiment can prevent cracks, buckling, and other failures in the sensor substrate 21 due to stress concentration. As a result, the present embodiment can inhibit occurrence of breakage of the wiring (the second wiring line 26B and the third wiring lines 26C) provided on the connecting portion 23 and damage to the photodiode PD.

The configuration illustrated in FIGS. 1 to 6 is merely exemplary, and the configuration of the sensor substrate 21, the photodiode PD, and other components can be changed as appropriate. For example, the shape of the notch 22 of the sensor substrate 21 is not limited to the configuration illustrated in FIG. 4. The radius of curvature (curvature) of the first portion 22a of the notch 22, for example, is not necessarily constant, and may vary along the arc. Alternatively, the notch 22 of the sensor substrate 21 does not necessarily have the second portion 22b. While the detection device 1 includes four photodiodes PD, the number of photodiodes PD may be three or less, or five or more. While the housing 200 illustrated in FIG. 2 has a ring shape with a curvature throughout its whole length along the circumference, the present embodiment is not limited thereto. The housing 200 may have a flat region in a partial region of the circumference, for example, in a portion provided with the light source 60.

### First Modification

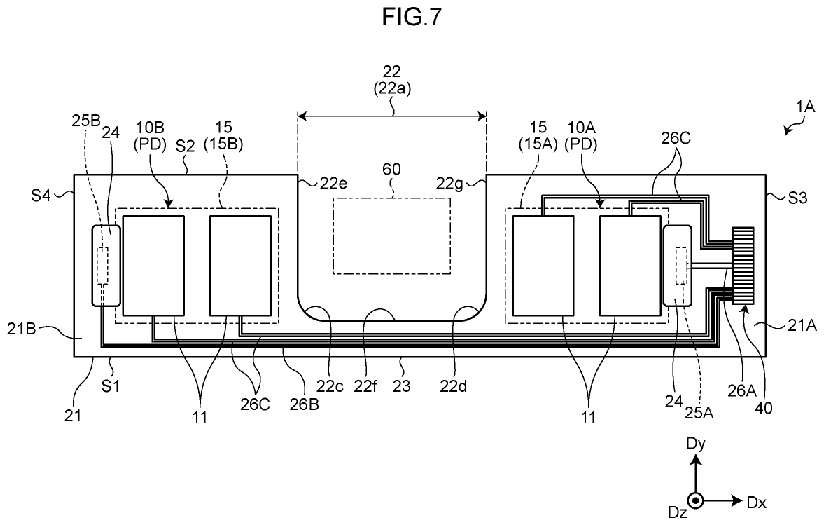

FIG. 7 is a plan view illustrating a configuration example of the sensor substrate according to a first modification of the first embodiment. In the following description, the same components as those described in any one of the embodiments described above are denoted by the same reference numerals, and the description thereof will not be repeated.

As illustrated in FIG. 7, in a detection device 1A according to the first modification, the notch 22 of the sensor substrate 21 has arc-shaped portions with a curvature at the corners. More specifically, the notch 22 has curved portions 22c and 22d and linear portions 22e, 22f, and 22g. The curved portions 22c and 22d each have an arc shape with a curvature. The curved portions 22c and 22d couple the linear portions 22e, 22f, and 22g adjacently formed along the outer periphery of the notch 22.

The linear portion 22e extends in the second direction Dy, with one end coupled to the second side S2 of the sensor substrate 21 and the other end coupled to the curved portion 22c. The linear portion 22f extends in the first direction Dx, with one end coupled to the curved portion 22c and the other end coupled to the curved portion 22d. The linear portion 22g extends in the second direction Dy, with one end coupled to the curved portion 22d and the other end coupled to the second side S2 of the sensor substrate 21.

In the first modification, the arc-shaped curved portions 22c and 22d with a curvature are formed at the corners of the notch 22 where stress concentration is likely to occur. This configuration can effectively reduce the generation of stress in the sensor substrate 21. In the first modification, the area of the notch 22 can be increased to secure a space for disposing the light source 60.

While the linear portions 22e and 22g of the notch 22 according to the first modification are directly coupled to the second side S2 of the sensor substrate 21, the present modification is not limited thereto. For example, the notch 22 may have the second portion 22b between each of the linear portions 22e and 22g and the second side S2 as in the first embodiment.

### Second Embodiment

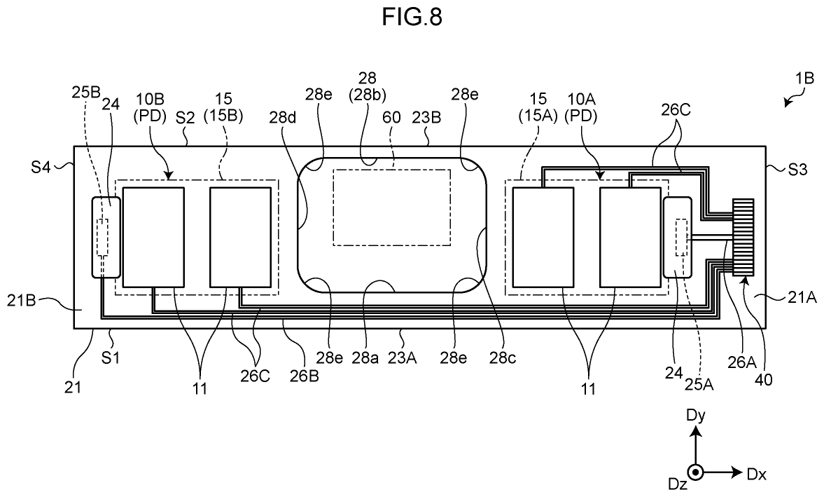

FIG. 8 is a plan view illustrating a configuration example of the sensor substrate according to a second embodiment. In the second embodiment and a second modification described below, the configurations of the first optical sensor 10A and the second optical sensor 10B are the same as those in the first embodiment described above (refer to FIGS. 5 and 6), and redundant description thereof is omitted.

As illustrated in FIG. 8, in a detection device 1B according to the second embodiment, the sensor substrate 21 has an opening 28. The opening 28 is formed between the region of the sensor substrate 21 provided with the first optical sensor 10A and the region provided with the second optical sensor 10B in the first direction Dx. The opening 28 is formed between the first side S1 and the second side S2 of the sensor substrate 21 in the second direction Dy. The opening 28 has a substantially rectangular shape with its long sides along the first side S1 and the second side S2, and the corners are smoothly curved in an arc shape.

The sensor substrate 21 has connecting portions 23A and 23B that are in contact with the opening 28 and extend in the first direction Dx. The connecting portion 23A is provided between the outer periphery of the opening 28 and the first side S1. The connecting portion 23B is provided between the outer periphery of the opening 28 and the second side S2. The region of the sensor substrate 21 provided with the first optical sensor 10A and the region provided with the second optical sensor 10B are integrally connected by the two connecting portions 23A and 23B.

More specifically, the outer periphery of the opening 28 has linear portions 28a, 28b, 28c, and 28d and four curved portions 28e. The linear portion 28a extends in a direction (first direction Dx) parallel to the first side S1 of the sensor substrate 21. The linear portion 28b extends in a direction (first direction Dx) parallel to the second side S2 of the sensor substrate 21. The linear portions 28c and 28d are formed between the linear portions 28a and 28b and extend in a direction (second direction Dy) parallel to the third side S3 and the fourth side S4, respectively, of the sensor substrate 21.

The four curved portions 28e each have an arc shape with a curvature. The four curved portions 28e are formed between the linear portions 28a, 28b, 28c, and 28d adjacently formed along the outer periphery of the opening 28. As a result, the adjacent linear portions 28a, 28b, 28c, and 28d are smoothly coupled by the curved portions 28e.

The light source 60 is disposed in the region overlapping the opening 28. The opening 28 is formed to have a length in the first direction Dx longer than the length of the light source 60. The opening 28 is formed to have a length in the second direction Dy longer than the length of the light source 60 and shorter than the length (width) of the sensor substrate 21. With this configuration, the opening 28 of the sensor substrate 21 can secure a space for disposing the light source 60.

The sensor substrate 21 according to the present embodiment has the arc-shaped curved portions 28e with a curvature at the corners of the opening 28 where stress concentration is likely to occur. With this configuration, the detection device 1B according to the second embodiment can reduce the generation of stress in the sensor substrate 21.

The connecting portion 23A of the sensor substrate 21 is formed between the linear portion 28a serving as the outer periphery of the opening 28 and the first side S1. The connecting portion 23B of the sensor substrate 21 is formed between the linear portion 28b serving as the outer periphery of the opening 28 and the second side S2. With this configuration, the detection device 1B according to the second embodiment can suppress twisting of the sensor substrate 21, for example, when the sensor substrate 21 is deformed into a ring shape along the housing 200.

The first width of the connecting portion 23A of the sensor substrate 21 in the second direction Dy is larger than the second width of the connecting portion 23B of the sensor substrate 21 in the second direction Dy. With this configuration, the sensor substrate 21 can secure a space for providing the second wiring 26B and the third wiring 26C.

### Second Modification

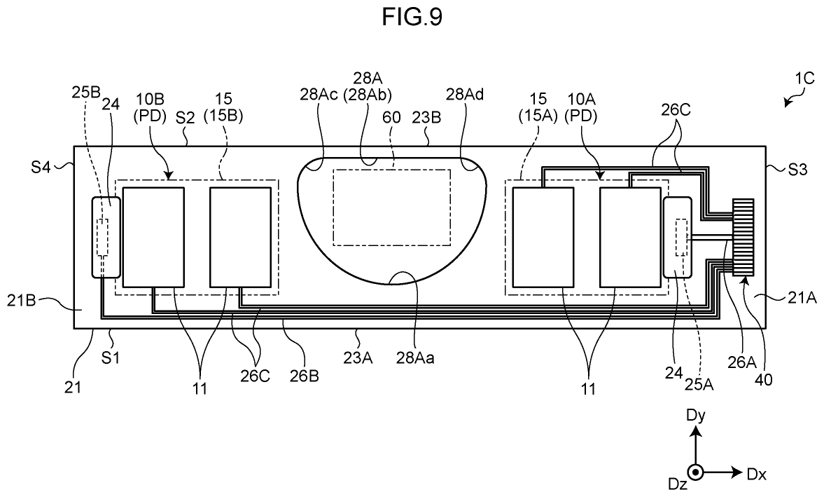

FIG. 9 is a plan view illustrating a configuration example of the sensor substrate according to a second modification of the second embodiment. As illustrated in FIG. 9, in a detection device 1C according to the second modification, the outer periphery of an opening 28A facing the first side S1 has an arc shape.

More specifically, the outer periphery of the opening 28A has a first curved portion 28Aa, a linear portion 28Ab, and two second curved portions 28Ac and 28Ad. The linear portion 28Ab extends in a direction (first direction Dx) parallel to the second side S2 of the sensor substrate 21. The first curved portion 28Aa is positioned closer to the first side S1 than the linear portion 28Ab and has an arc shape with a curvature throughout its whole length.

The two second curved portions 28Ac and 28Ad have an arc shape with a radius of curvature smaller than that of the first curved portion 28Aa and couple the linear portion 28Ab to the first curved portion 28Aa. The second curved portion 28Ac couples one end (left end in FIG. 9) of the linear portion 28Ab to one end (left end in FIG. 9) of the first curved portion 28Aa. The second curved portion 28Ad couples the other end (right end in FIG. 9) of the linear portion 28Ab to the other end (right end in FIG. 9) of the first curved portion 28Aa.

In the outer periphery of the opening 28A according to the present modification, the first curved portion 28Aa close to the first side S1 (connecting portion 23A) of the sensor substrate 21 is formed into an arc shape with a curvature throughout its whole length. This configuration can distribute the stress along the first curved portion 28Aa and reduce stress concentration at the center of the connecting portion 23A in the first direction Dx (portion with the smallest width in the second direction Dy). Therefore, the present modification can inhibit occurrence of breakage of the second wiring line 26B and the third wiring lines 26C provided to the connecting portion 23A.

The shape, size, width, and the like of the openings 28 and 28A described in the second embodiment and the second modification are given by way of example only and may be modified as appropriate. For example, the opening 28 according to the second embodiment has a substantially rectangular shape with its long sides along the first direction Dx. The shape of the opening 28 is not limited thereto, and may be other shapes, such as a substantially square shape.

While the preferred embodiments of the present disclosure have been described above, the present disclosure is not limited to the embodiments described above. The content disclosed in the embodiments is merely an example, and can be variously modified within the scope not departing from the gist of the present disclosure. Any modifications appropriately made within the scope not departing from the gist of the present disclosure also naturally belong to the technical scope of the present disclosure. At least one of various omissions, substitutions, and changes of the components can be made without departing from the gist of the embodiments and the modifications thereof described above.

What is claimed is:

1. A detection device comprising:
a housing having a ring shape;
a sensor substrate provided in the housing and curved along the shape of the housing;
a first optical sensor and a second optical sensor provided to the sensor substrate;
a light source disposed in the housing; and
a plurality of wiring lines coupling each of the first optical sensor and the second optical sensor to a terminal part provided to the sensor substrate, wherein
the sensor substrate has a first side extending in a first direction, a second side parallel to the first side, and a notch formed by cutting out the second side when the sensor substrate is developed into a flat shape,
the notch includes a first portion having an arc shape with a curvature throughout its whole length and is formed between the first optical sensor and the second optical sensor in the first direction, and
the light source is disposed in a region overlapping the notch.

2. The detection device according to claim 1, wherein
the notch includes a second portion formed between the first portion and the second side, and
the second portion extends in a direction different from a tangential direction at an end of the first portion.

3. The detection device according to claim 1, wherein
each of the first optical sensor and the second optical sensor is configured with a lower electrode, a lower buffer layer, an active layer, an upper buffer layer, and an upper electrode stacked on the sensor substrate in the order as listed, and
the wiring lines are coupled to the lower electrodes.

4. The detection device according to claim 1, wherein the housing has a ring shape capable of being worn on a finger.

5. A detection device comprising:
a housing having a ring shape;
a sensor substrate provided in the housing and curved along the shape of the housing;
a first optical sensor and a second optical sensor provided to the sensor substrate;
a light source disposed in the housing; and
a plurality of wiring lines coupling each of the first optical sensor and the second optical sensor to a terminal part provided to the sensor substrate, wherein
the sensor substrate has a first side extending in a first direction, a second side parallel to the first side, and an opening formed between the first side and the second side when the sensor substrate is developed into a flat shape,
a first width of the sensor substrate between the first side and an outer periphery of the opening facing the first side is larger than a second width of the sensor substrate between the second side and an outer periphery of the opening facing the second side,
the opening is formed between the first optical sensor and the second optical sensor in the first direction, and
the light source is disposed in a region overlapping the opening.

6. The detection device according to claim 5, wherein the outer periphery of the opening facing the first side has an arc shape.

7. The detection device according to claim 5, wherein
each of the first optical sensor and the second optical sensor is configured with a lower electrode, a lower buffer layer, an active layer, an upper buffer layer, and an upper electrode stacked on the sensor substrate in the order as listed, and
the wiring lines are coupled to the lower electrodes.

8. The detection device according to claim 5, wherein the housing has a ring shape capable of being worn on a finger.
